(19) United States
(12) Patent Application Publication
(10) Pub. No.: US 2018/0158664 A1
(43) Pub. Date: Jun. 7, 2018

(54) Mobility Electrophoresis Separation Device, Operating Method Thereof, and Interface Between Liquid Chromatography and Mass Spectrometry

(71) Applicant: Beijing Institute Of Technology, Beijing (CN)

(72) Inventors: Wei Xu, Beijing (CN); Muyi He, Beijing (CN); Pan Luo, Beijing (CN)

(21) Appl. No.: 15/832,537

(22) Filed: Dec. 5, 2017

(30) Foreign Application Priority Data
Dec. 5, 2016 (CN) 201611104754.7

(51) Int. Cl. H01J 49/16; G01N 27/447; G01N 30/72; G01N 33/68; G01N 30/46; G01N 27/62; H01J 49/04

(52) U.S. Cl. CPC H01J 49/167; G01N 27/44769; G01N 30/7266; H01J 49/0404; G01N 30/463; G01N 27/622; G01N 33/68

(57) ABSTRACT

The present invention provides a mobility electrophoresis separation device, its operating method, and an interface between liquid chromatography and mass spectrometry. The mobility electrophoresis separation device comprises a separation capillary, a syringe pump for injecting a buffer solution, a syringe for injecting a sample solution, and two electrodes disposed apart from each other on either side of the separation capillary. A sample solution is injected by a syringe at a position of the capillary channel, and a buffer solution is injected into the capillary channel upstream the first position, and carries the sample solution to flow downstream. While the mixed liquid flows through the capillary, an electric field is applied in the direction of the flow. Different ions in the sample are thus separated in the flow due to their different velocities traveling in the flow.

### CROSS-REFERENCE TO RELATED APPLICATION

This application claims priority to Chinese Patent Application No. 201611104754.7, filed Dec. 5, 2016, the disclosure of which is incorporated herein by reference in its entirety

### TECHNICAL FIELD

The present invention relates to the field of mass spectrometry and, more particularly, to a mobility electrophoresis separation device and the associated control method, as well as an interface between liquid chromatography and mass spectrometry.

### BACKGROUND

Mass spectrometry is an analytical method of identifying substances in a sample by separating and detecting different components in the sample according to their different mass to charge ratios (m/z). Because of its high specificity and sensitivity, mass spectrometry is becoming increasingly important in the field of biological analysis.

The basic principle of mass spectrometry is to ionize the components of a sample in the ion source, generate ions with different mass-to-charge ratios, and enter the mass analyzer in the form of ion beams. For the detection of liquid samples, the most commonly used ion source is the electrospray ion source. When using electrospray ionization source to detect mixed samples, ionization competition may occur between the components to be detected, as well as between the components to be detected and impurities, so that the components with low abundance and low ionization efficiency are not easy to be detected.

### SUMMARY OF THE INVENTION

In order to overcome the above technical problems, the present invention provides a mobility electrophoresis separation device and a control method, as well as an interface with liquid chromatography and mass spectrometry, to realize the separation of components of a complex sample system and enhance the separation effectiveness.

In order to achieve the above object, the present invention provides a mobility electrophoresis separation device comprising:

a separating capillary, where one end of the separation capillary is an electrospray tip and the other end is a buffer solution injection end;

a syringe pump, connected to the buffer solution injection end;

a syringe, connected to the separation capillary at a position close to the syringe pump;

a separation electrode, connected to the syringe pump, or connected to the separation capillary at a position close to the syringe pump; and

a ground electrode, connected to the separation capillary at a position close to the electrospray tip.

In some embodiments, the mobility electrophoresis separation device further comprises:

a first multi-way valve, through which the syringe pump and the syringe are connected to the separation capillary; and

a second multi-way valve, through which the ground electrode is connected to the separation capillary.

In some embodiments, the separation electrode is connected to the separation electrode through the first multi-way valve.

In some embodiments, the mobility electrophoresis separation device further comprises:

an auxiliary capillary, which connects the separation capillary through the second multi-way valve to introduce the auxiliary buffer solution into the separation capillary, or export the waste liquid.

The present invention also provides an interface between liquid chromatography and mass spectrometry comprising:

any one of the mobility electrophoresis separation devices as described above;

a liquid chromatography apparatus, wherein the sample outlet of the liquid chromatography apparatus is connected to the syringe of the mobility electrophoresis separation device.

The present invention also provides a method for controlling the mobility electrophoresis separation device comprising:

turning on the syringe pump to inject the buffer solution into the separation capillary at a predetermined flushing pressure;

closing the syringe pump after the first time period;

injecting the sample into the separation capillary at a predetermined injection pressure;

stopping the injection of the syringe after the second time period, turning on the syringe pump again to inject the buffer solution into the separation capillary at a predetermined separation pressure, applying separation voltage through the separation electrode, and applying spray voltage to the electrospray tip.

In some embodiments, the flushing pressure is in the range of 1 to 1000 mbar, the injection pressure is in the range of 10 to 100 mbar, the separation pressure is in the range of 1 to 200 mbar, the first time period is in the range of 4 to 6 minutes and the second time period is in the range of 1 to 10 seconds.

The present invention also provides a mobility electrophoresis separation method, which separates ions in a liquid channel based on electric fields and a liquid flow. In some embodiments, a mobility electrophoresis separation method comprises: creating a flow of a liquid containing different species of ions in a flow channel; and applying an electric field along the flow channel, thereby separating the different species of ions based on their different traveling velocities in the liquid flow under the applied electric field. In one embodiment, creating the flow comprises injecting a sample solution containing the different species of ions into a first position of the flow channel; and introducing a carrier solution upstream of the first position in the flow channel, thereby causing the carrier solution to flow through the first position and carrying the different species of ions in the sample solution downstream from the first position. In the method, introducing the buffer solution can be performed at a predetermined pressure, to obtain a desired buffer solution flow speed in the capillary. The flow channel can be a capillary channel.

According to the mobility electrophoresis separation device, the control method and the interface between liquid chromatography and mass spectrometry of the present invention, the mobility electrophoresis separation device comprises a separation capillary, a syringe pump, a syringe, a separation electrode and a ground electrode, wherein one end of the capillary is an electrospray tip and the other end is a buffer solution injection end; the syringe pump is connected to the buffer solution injection end; the syringe is connected to the separation capillary at a position close to the syringe pump; the separation electrode is connected to the separation capillary at a position close to the syringe pump; and the ground electrode is connected to the separation capillary at a position close to the electrospray tip. The present invention utilizes a syringe pump to inject buffer solution to rinse the sample in the separation capillary while applying a separate electric field so that the components in the sample are sufficiently separated in the separation capillary to enhance the separation effect with a fast separation rate and the product structure is simple and easy to operate.

### BRIEF DESCRIPTION OF THE DRAWINGS

FIG. 1 is a diagram illustrating the mobility electrophoresis principle;

FIGS. 2-1 to 2-2 show theoretical trajectories and chromatograms of ions under different electric fields.

FIG. 3 is a structural diagram of a mobility electrophoresis separation device according to an example of the present invention;

FIGS. 4-1 to 4-4 are structural diagrams of four other mobility electrophoresis separation devices provided in the examples of the present invention;

FIG. 5 is a flow diagram of a control method for a mobility electrophoresis separation device according to an example of the present invention;

FIGS. 6-1 and 6-2 are comparative diagrams of the sample migration time when different pressures are applied in the examples of the present invention;

FIGS. 7-1 and 7-2 are comparison diagrams of the sample migration time when different separation voltages are applied in the examples of the present invention;

FIGS. 8-1 and 8-2 are comparison diagrams of the sample migration time when the length of the separation capillary is different in the examples of the present invention;

FIGS. 9-1 and 9-2 are comparison diagrams of the sample migration time when the viscosity coefficients of the buffer solution are different in the examples of the present invention;

FIG. 10 shows comparison diagrams of the sample migration time when the numbers of charges in the sample are different in the examples of the present invention;

FIG. 11 shows comparison diagrams of a sample migration time when the sample geometry is different in the examples of the present invention;

FIGS. 12-1 and 12-2 are diagrams showing the effect of separating various mixed samples using the examples of the present invention.

### DETAILED DESCRIPTION OF EMBODIMENTS

The examples of the present invention are illustrated below with the aid of the drawings. Elements and features described in one drawing or one embodiment of the present invention may be combined with elements and features described in one or more other drawings or embodiments. It should be noted that for the purpose of clarity, expressions and descriptions of components or processes that are well known to those skilled in this art and are not pertinent to the present invention are omitted from the drawings and description.

The present invention is further described below with the aid of the drawings.

The examples of the present invention propose a mobility electrophoresis theory and provides a mobility electrophoresis separation device and control method thereof on the basis of this theory. Mobility electrophoresis refers to carrying out separation on the basis of the gas phase ion mobility spectrum in combination with the differential motion. In the examples of the present invention, the mixed sample moved along with a carrier undergoes differential motion under the action of an electric field. In the case of positively charged substances, the reverse electric field impedes the migration of positively charged substances. According to the examples of the present invention, the mobility electrophoresis separation can be used to further separation analysis after chromatographic separation. The chromatography separation is mainly based on differential polarity of the sample, while the mobility electrophoresis separation device can be used for further separation of the sample based on the different charge properties of the components of the sample.

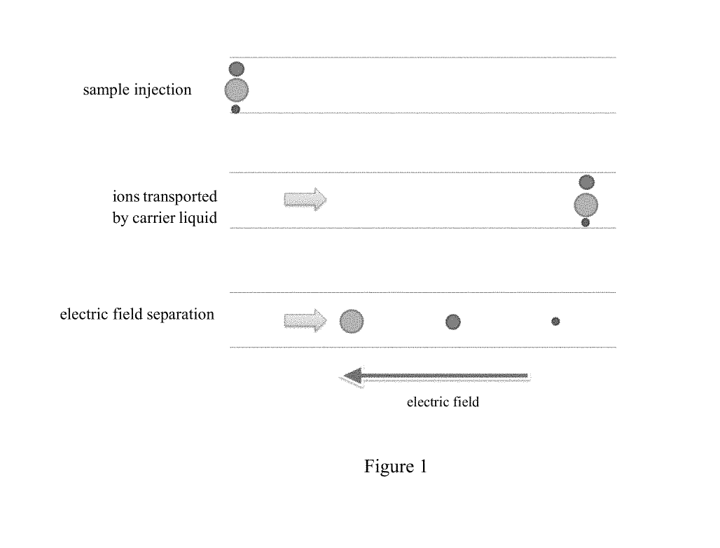

The principle of mobility electrophoresis is shown in FIG. 1. After a sample of mixed components is injected into the separation channel, a buffer solution is injected. The buffer (or carrier) solution carries the sample forward. A separation voltage is then applied, and the differences in the charges and equivalent radii of the different components result in different velocity, thereby causing separation of these components.

The mixed sample in the separation channel, in the traction of advection incompressible carrier will move uniformly. The equilibrium speed of each component is the same as the carrier velocity v_{carrier}. If the mixed sample can be dissociated into ions, the ions are subjected to an electric field force F_{E} in a direction opposite to the carrier motion by applying a reverse electric field, thereby causing a relative motion between the ions and the carrier. This process causes the components to be subjected to stress F_{f}. F_{E} and F_{f} can be obtained by the following formula, respectively:

F_{E}=qE

F_{f}=6πηrv

Where E is the separation electric field intensity, q is the ion charge amount, η is the viscosity coefficient of the buffer solution, r is the equivalent radius of the subject ion, and v is the velocity of the subject ion relative to the carrier. When F_{E} and F_{f} are equal, the relative velocity v between the subject ion and the carrier is constant, and the apparent velocity v_{E} is:

v_{E}=v_{carrier}+v

The apparent velocity of the ions is related to their respective charges and equivalent radii. Different ions have different apparent velocities. The time of different ions traveling through a length L of the separation channel is different, thereby causing the separation of the ions.

The apparent displacement S of an ion can be represented as the following differential equation:

m\ddot{S}+6πηr\dot{S}=qE=qU/L.

wherein U is the separation voltage.

Through numerically solving the differential equation, the time of migration distance L of different ions can be obtained.

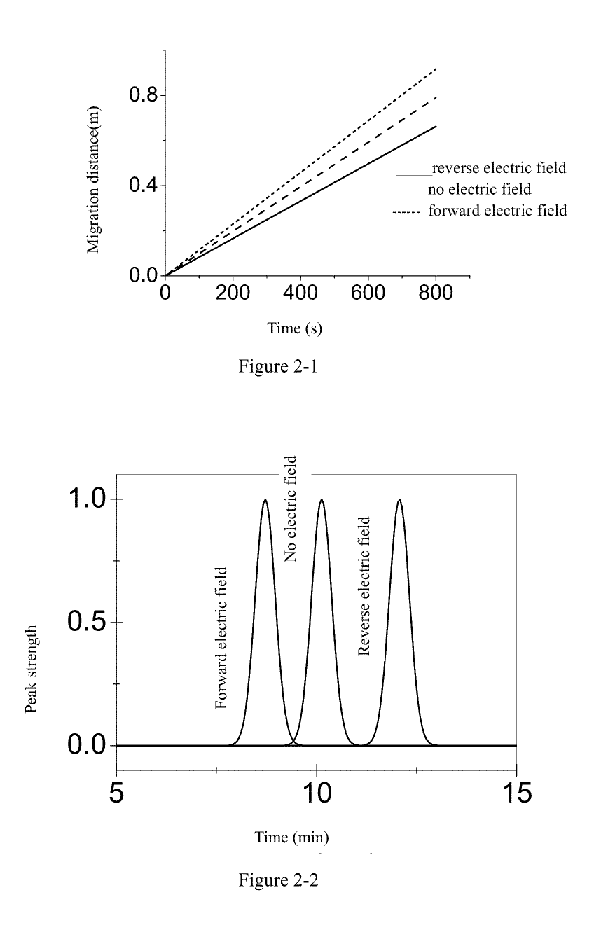

FIGS. 2-1 and FIG. 2-2 show the theoretical trajectories and chromatograms of ions under different electric fields. The calculated conditions are given below: the separation channel length is 60 cm, the channel inner diameter is 75 μm, the potential is ±10 kV, the pressure is 30 mbar, the viscosity coefficient is 0.89 mPa·S, the sample has a relative molecular weight of 433, with one positive charge and an equivalent radius of 1 nm. FIG. 2-1 shows the trajectories of ions at +10 kV, 0V, and −10 kV. The forward electric field causes the ions to peak earlier and the reverse voltage delays the peaking of the ions. If the reverse electric field continues to increase, the ions may not peak. FIG. 2-2 is the theoretical chromatogram, in which the forward electric field peak time is the shortest and the negative electric field peak time is the longest.

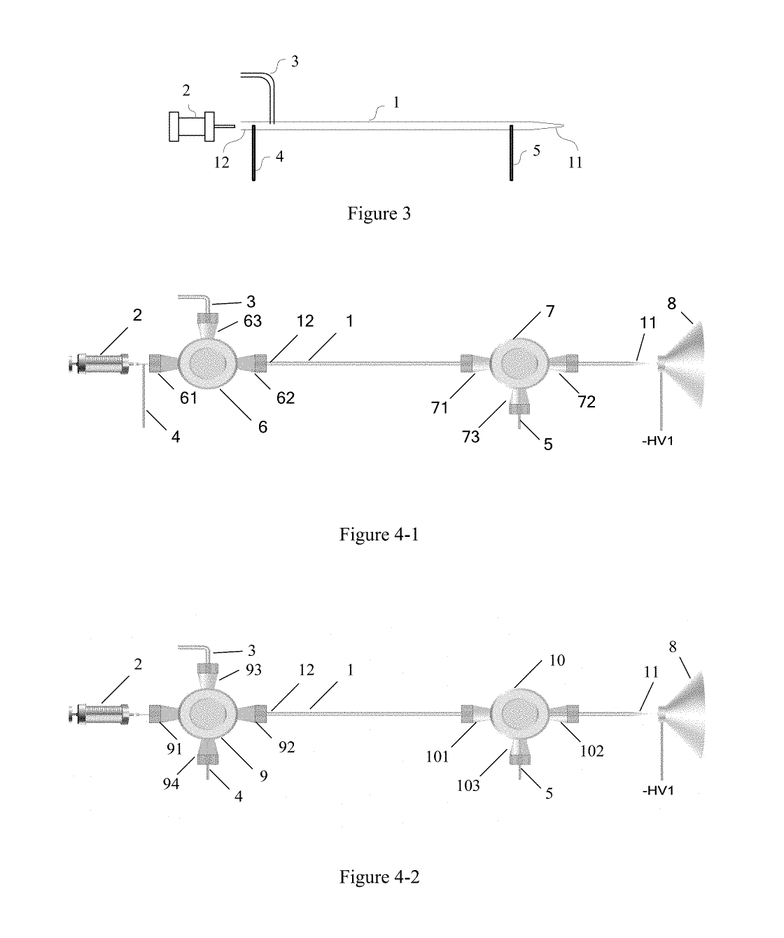

The example of the present invention provides a mobility electrophoresis separation device comprising a separation capillary 1, a syringe pump 2, a syringe 3, a separating electrode 4 and a ground electrode 5 as shown in FIG. 3.

One end of the separation capillary 1 is an electrospray tip 11 and the other end is an injection end 12 configured to receive a buffer solution. The syringe pump 2 is connected to the buffer solution injection end 12 of the separation capillary 1. The syringe 3 is connected to the separation capillary 1 at a position close to the syringe pump 2. The separation electrode 4 is connected to the syringe pump, or is connected to the separation capillary 1 at a position close to the syringe pump 2. The ground electrode 5 is connected to the separation capillary 1 at a position close to the electrospray tip 11.

The syringe pump 2 is turned on to inject the buffer solution into the separation capillary 1 at a predetermined constant pressure. After a predetermined period of time, the syringe pump 2 is closed. The syringe 3 then starts to inject the sample into the separation capillary 1. After stopping the injection of the syringe 3, the syringe pump 2 is turned on again to inject the buffer solution into the separation capillary 1, and the separation voltage is applied through the separation electrode 4, and the spray voltage is applied to the electrospray tip 11.

The mobility electrophoresis separation device according to the present invention utilizes a syringe pump to inject buffer solution to rinse the sample in the separation capillary while applying a separate electric field, so that the components in the sample are sufficiently separated in the separation capillary to enhance the separation effect with a fast separation rate. The device has a rather simple structure, and is easy to operate.

The mobility electrophoresis separation device provided by the example of the present invention further includes a first multi-way valve and a second multi-way valve. The syringe pump and syringe are connected to the separation capillary through the first multi-valve. The ground electrode is connected to the separation capillary through the second multi-way valve.

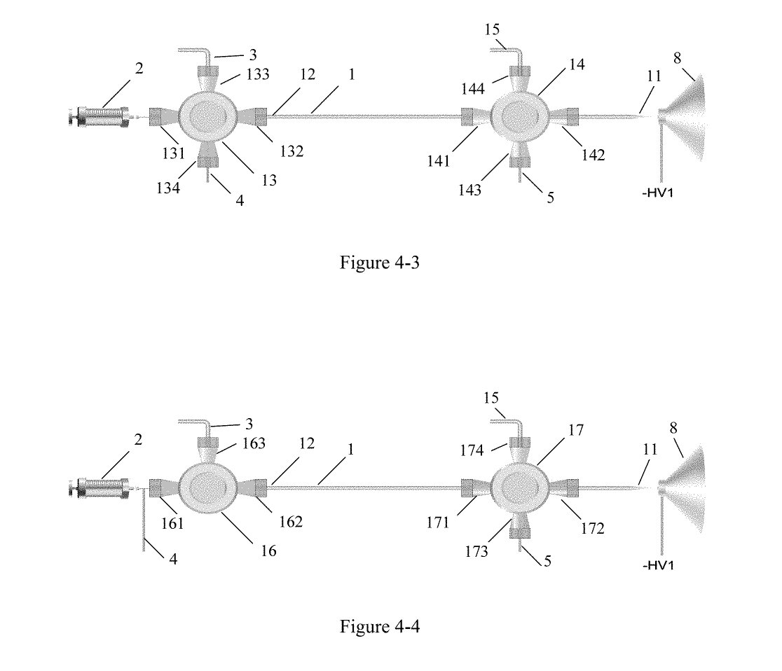

A particular embodiment of the mobility electrophoresis separation device provided by the example of the present invention is shown in FIG. 4-1. The first multi-way valve and the second multi-way valve are specifically three-way valve 6 and three-way valve 7.

The syringe pump 2, the buffer solution injection end 12 of the separation capillary 1 and the syringe 3 are connected to three ports 61, 62, 63 of the three-way valve 6, respectively, through this connection, the syringe pump 2 and the syringe 3 inject the buffer solution and the sample into the separation capillary 1 through the valve 6, respectively. The separation electrode 4 may be provided between the syringe pump 2 and the three-way valve 6. The separation capillary 1 extending from the three-way valve 6 enters the first port 71 of the three-way valve 7, and the electrospray tip 11 extends from the second port 72 of the three-way valve 7. The ground electrode 5 is connected to the third port 73 of the three-way valve 7.

A particular embodiment of the mobility electrophoresis separation device provided by the example of the present invention is shown in FIG. 4-2. The first multi-way valve and the second multi-way valve are specifically four-way valve 9 and three-way valve 10.

The syringe pump 2, the buffer solution injection end 12 of the separation capillary 1, the syringe 3, and the separating electrode 4 are connected to four ports 91, 92, 93, 94 of the four-way valve 9, respectively, and through this connection, the syringe pump 2 and the syringe 3 inject the buffer solution and the sample into the separation capillary 1 through the four-way valve 9, respectively, and the separation electrode 4 supplies the separation voltage to the mixture of the buffer solution and the sample. The separation capillary 1 extending from the four-way valve 9 enters the first port 101 of the three-way valve 10, and the electrospray tip 11 extends from the second port 102 of the three-way valve 10. The ground electrode 5 is connected to the third port 103 of the three-way valve 10.

A particular embodiment of the mobility electrophoresis separation device provided by the examples of the present invention is shown in FIG. 4-3. The first multi-way valve and the second multi-way valve are specifically four-way valves 13 and four-way valves 14, respectively. An auxiliary capillary 15 is also included in FIG. 4-3.

The syringe pump 2, the buffer solution injection end 12 of separation capillary 1, the syringe 3, and the separating electrode 4 are connected to four ports 131, 132, 133 and 134 of the four-way valve 13, respectively, and through this connection, the syringe pump 2 and the syringe 3 inject the buffer solution and the sample into the separation capillary 1 through the four-way valve 13, respectively, and the separation electrode 4 supplies the separation voltage to the mixture of the buffer solution and the sample. The separation capillary 1 extending from the four-way valve 13 enters the first port 141 of the four-way valve 14, and the electrospray tip 11 extends from the second port 142 of the four-way valve 14. The ground electrode 5 is connected to the third port 143 of the four-way valve 14, and the auxiliary capillary 15 is connected to the fourth port 144 of the four-way valve 14. The auxiliary capillary 15 may be used to provide ionization assisted spray buffer solution to the separation capillary 1, or may be used to absorb the waste liquid from the separation capillary 1.

A particular embodiment of the mobility electrophoresis separation device provided by the examples of the present invention is shown in FIG. 4-4. The first multi-way valve and the second multi-way valve are specifically three-way valves 16 and four-way valves 17, respectively. The auxiliary capillary 15 is also included in FIG. 4-4.

The syringe pump 2, the buffer solution injection end 12 of the separation capillary 1 and the syringe 3 are connected to three ports 161, 162, 163 of the three-way valve 16, respectively, through this connection, the syringe pump 2 and the syringe 3 inject the buffer solution and the sample into the separation capillary 1 through three-way valve 16. The separation electrode 4 may be provided between the syringe pump 2 and the three-way valve 16. The separation capillary 1 extending from the three-way valve 16 enters the first port 171 of the four-way valve 17, and the electrospray tip 11 extends from the second port 172 of the four-way valve 17. The ground electrode 5 is connected to the third port 173 of the four-way valve 17, and the auxiliary capillary 15 is connected to the fourth port 174 of the four-way valve 17. The auxiliary capillary 15 may be used to provide ionization assisted spray buffer solution to the separation capillary 1, or may be used to absorb the waste liquid from the separation capillary 1.

In FIGS. 4-1 to 4-4, the electrospray tip 11 may be connected to the mass spectrometer inlet 8. A spray voltage −HV1 is applied to the electrospray tip 11 while the separation voltage +/−HV2 is applied to the separation electrode 4.

In this mobility electrophoresis separation device, the separation capillary is kept as straight as possible. To this end, suitable three-way valve and four-way valve should be selected so that the separation of capillary can run through the three-way valve and four-way valve.

In another example of the examples of the present invention, a detection window may be opened at the separation capillary to assist in detecting the sample separation process by means of an optical detection method.

In another example of the examples of the present invention, multimodal separation can also be achieved by varying the buffer solution system, e.g., adding a MS-compatible surfactant in the buffer solution to form a micelle can be used for the separation of the neutral substances and improvement of the separation of charged substances.

The examples of the present invention also provide an interface between liquid chromatography and mass spectrometry (LC-MS) including the mobility electrophoresis separation devices as described above and a liquid chromatographic apparatus, wherein the sample outlet of the liquid chromatographic apparatus is connected to the syringe of the mobility electrophoresis separation device. The liquid chromatographic apparatus is used for sample separation based on the polarity of the sample. The product with similar polarity then flows into the syringe, and injected into the mobility electrophoresis separation device for further separation based on their different electrophoresis characteristics.

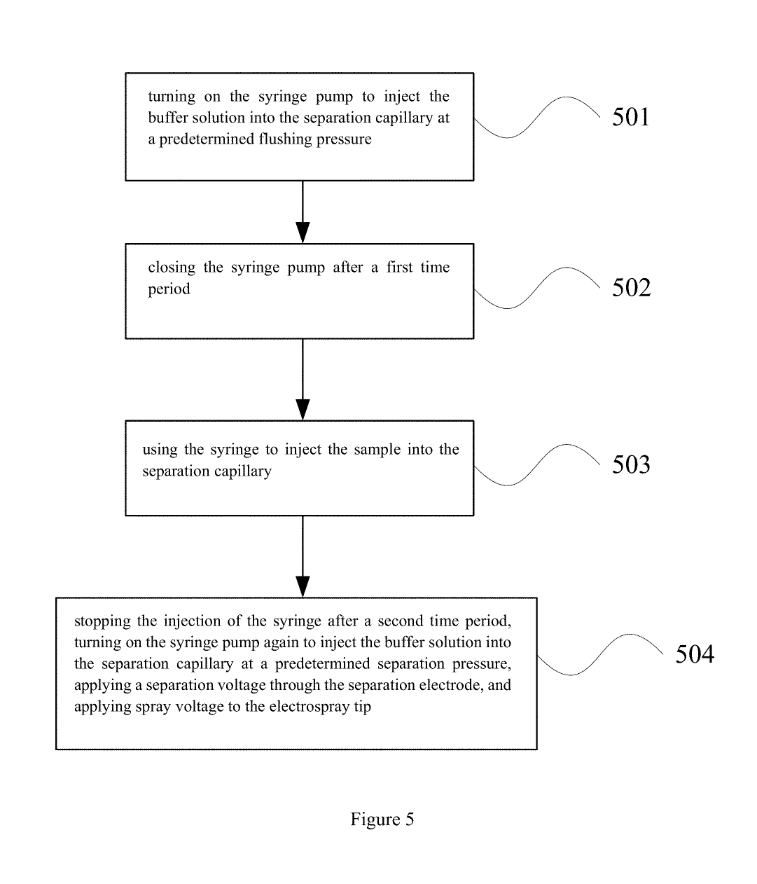

The example of the present invention also provides a method of operating the mobility electrophoresis separation device comprising, as shown in FIG. 5:

501, turning on the syringe pump to inject the buffer solution into the separation capillary at a predetermined flushing pressure;

The separation capillary can use a variety of internal and external diameters, the spray tip can have a diameter range of 3 μm˜50 μm. The buffer solution may be aqueous methanol solution (containing 0.1% formic acid (w/w)), or ammonium acetate solution, or ammonium formate solution.

502, closing the syringe pump after a first time period;

The predetermined time period can be 4 to 6 minutes, and may be 5 minutes in a specific operation.

503, using the syringe to inject the sample into the separation capillary at a predetermined injection pressure;

504, stopping the injection of the syringe after a second time period, turning on the syringe pump again to inject the buffer solution into the separation capillary at a predetermined separation pressure, applying a separation voltage through the separation electrode, and applying spray voltage to the electrospray tip.

The separation pressure can continue for 0.1 to 60 minutes, depending on the peak time.

The flushing pressure can be 1 to 1000 mbar, the injection pressure can be 10 to 100 mbar, the separation pressure can be 1 to 200 mbar, the first time period can be 4 to 6 minutes, and the second time period can be 1 to 10 seconds.

In practical applications, the flushing pressure can be 900 mbar, the injection pressure is 50 mbar, the separation pressure is specifically 30 mbar, the first time period is specifically 5 minutes, and the second time period is specifically 5 seconds.

The output range of the separation voltage is from 0 to +/−30 kV.

After the syringe pump is turned on again to inject the buffer solution, the injected sample in the separation capillary moves to the electrospray tip under the load of the buffer solution, at this time the separation voltage is supplied through the separation electrode so that the mixed components of the sample in the separation capillary are separated.

The syringe pump is used to inject the buffer solution into the separation capillary; this flow-type operation facilitates miniaturization of the separation capillary, reduces the size of the device and is easy to operate. Furthermore, the closed capillary channel during the flow operation avoids the evaporation of the liquid, provides a precisely repeatable passage through which the solution flows and a safe environment.

Optionally, an ionization assisted spray buffer solution may also be provided to the separation capillary through the auxiliary capillary, and the waste liquid may also be absorbed from the separation capillary by means of an auxiliary capillary. The spray buffer solution may be a methanol or acetonitrile solution.

The method of controlling the mobility electrophoresis separation device provided by the example of the present invention uses a syringe pump to inject buffer solution to rinse the sample in the separation capillary while applying a separate electric field, so that the components in the sample are sufficiently separated in the separation capillary, with improved separation effect, fast separation speed, simple product structure and easy operation.

As described above, the apparent velocity v_{E} is related to the parameters such as the separation electric field intensity E, the ion charge amount q, the viscosity coefficient η, the equivalent radius of the ions r, and the change of these parameters will affect the ion migration time. The specific analysis is carried out by the following Examples 1 to 7.

### EXAMPLE 1

This example analyzes the effect of pressure on the migration time of the sample by simulation and experiments, respectively.

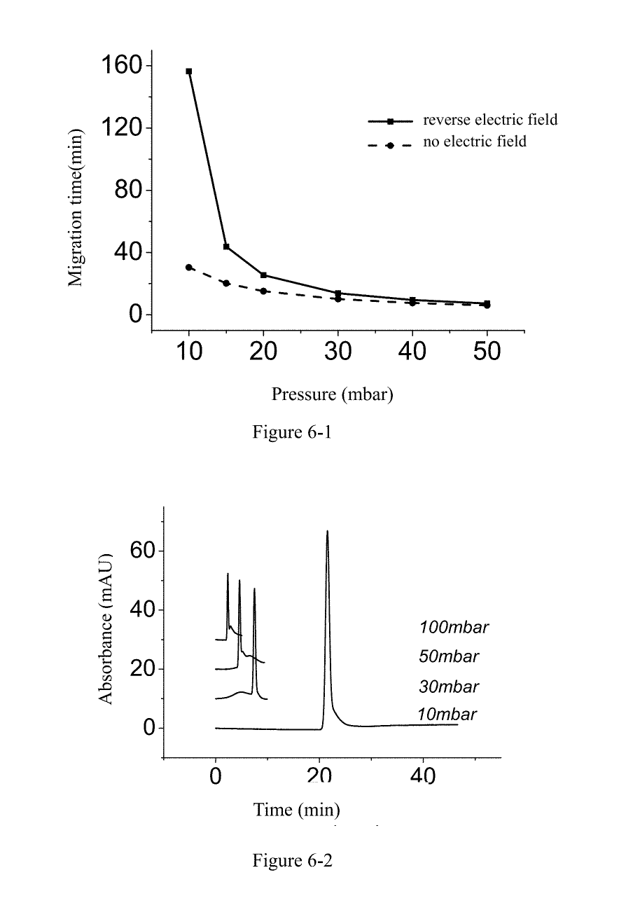

The simulation parameters are set as follows: capillary length 60 cm, tube inner diameter 75 μm, reverse separation voltage −10 kV or no separation voltage, viscosity coefficient 0.89 mPa·S, sample with relative molecular weight 1048 two positive charges and equivalent radius 1.2 nm. The separation pressures at both ends of the separation channel are 10 mbar, 15 mbar, 20 mbar, 30 mbar, 40 mbar, 50 mbar, respectively. The simulation results are shown in FIG. 6-1. It can be seen that the migration time of the substance is shortened, the migration time sharply shortens from 10 mbar to 15 mbar and the migration time is gradually shortened from 15 mbar to 50 mbar as the pressure at both ends of the separation channel increases and the reverse separation voltage is applied. Compared with the results of the migration without applying electric field, the smaller the separation pressure, the greater the substance migration time is affected by the electric field.

Experimental conditions: the sample was 1 mg/mL angiotensin II, the buffer solution was 20% methanol aqueous solution (containing 0.1% formic acid (w/w)), the capillary tube length was 40 cm, the tube inner diameter was 75 μm. Operation mode: the sample was injected under injection pressure of 50 mbar for 5 s, only a separation pressure was applied at both ends of the separation channel, no voltage was applied, the separation pressure was 10 mbar, 30 mbar, 50 mbar, 100 mbar, respectively. The experimental results are shown in FIG. 6-2. It can be seen that with the increase of air pressure, the greater the thrust on the sample by the buffer solution, the faster the movement becomes, so the sample migration time becomes shorter. In addition, the higher the pressure becomes, the lower the sample peak height and the peak also has longer tails.

The injection pressure and separation pressure can be provided by the same pneumatic device. The injection can also be done by voltage or siphon.

### EXAMPLE 2

This example analyzes the effect of separation voltage on sample migration time by simulation and experiments.

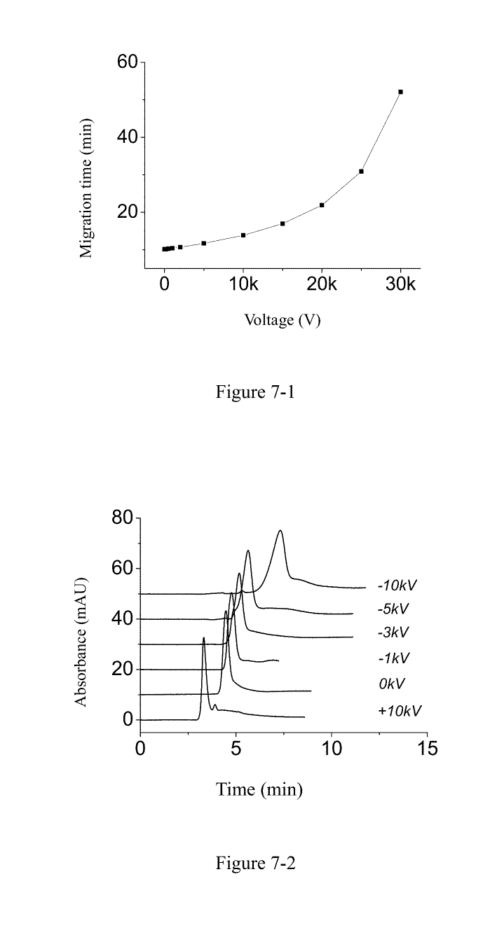

The simulation parameters were set as follows: capillary tube length 60 cm, tube inner diameter 75 μm, separation pressure of pumping buffer solution 30 mbar, viscosity coefficient 0.89 mPa·S, sample with relative molecular weight 1048, two positive charges and equivalent radius 1.2 nm. The reverse separation voltages were 0V, −100V, −200V, −500V, −1 kV, −2 kV, −5 kV, −10 kV, −15 kV, −20 kV, −25 kV, −30 kV, respectively. The results in FIG. 7-1 show that with the increase of the separation voltage, the substance migration time is gradually extended.

Experimental conditions: The experimental conditions of Example 1 are used, specifically: the sample was 1 mg/mL of angiotensin II, the buffer solution was 20% aqueous methanol solution (containing 0.1% formic acid), the capillary tube length was 60 cm, the tube inner diameter was 75 μm, a separation pressure of 50 mbar was selected to pump the buffer solution and a separation voltage at both ends of the separation channel was applied. The voltages of +10 kV, −1 kV, −3 kV, −5 kV, −10 kV were applied respectively, and the experimental results with applying these voltages or without applying the separation voltage were compared. The experimental results are shown in FIG. 7-2. +10 kV shortens the sample migration time to make the sample peak earlier. When applying a voltage reversed with air pressure, the migration time of the sample is prolonged as the voltage increases, and the peak width of the sample peak becomes wider and the peak height becomes lower.

### EXAMPLE 3

This example analyzes the effect of separation capillary length on sample migration time by simulation and experiments.

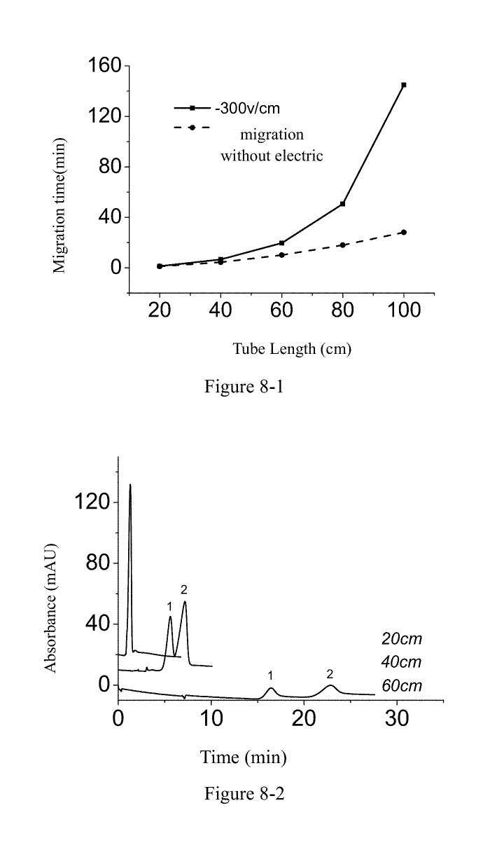

The simulation parameters were set as follows: the tube inner diameter was 75 μm, the reverse separation field strength was −300 V/cm, the separation pressure of pumping buffer solution was 30 mbar, the viscosity coefficient was 0.89 mPa·S, the sample had a relative molecular weight of 1048 with two positive charges and equivalent radius of 1.2 nm. The capillary tube lengths are 20 cm, 40 cm, 60 cm, 80 cm, 100 cm, respectively. The simulation results are shown in FIG. 8-1. It can be seen that with the increase of the capillary tube length, the substance migration time increases slowly and then increases rapidly.

Experimental conditions: The sample was a mixed solution of angiotensin II with a final concentration of 1 mg/mL and 1 mg/mL bradykinin, the buffer solution was 20% methanol aqueous solution (containing 0.1% formic acid (w/w)) and the capillary tube inner diameter was 75 μm. Operation mode: the injection was carried out under injection pressure of 50 mbar for 5 s, 50 mbar separation pressure and −410V/cm field strength were applied at both ends of the separation channel, the capillary tube length were 20 cm, 40 cm, 60 cm, respectively. The experimental results are shown in FIG. 8-2. It can be seen that with the increase of tube length, the longer the sample migration time becomes, the greater the separation between the components becomes, while the peak width increases. In this Figure, substance 1 is angiotensin II and substance 2 is bradykinin.

### EXAMPLE 4

This example analyzes the effect of the viscosity coefficient η of the buffer solution on the sample migration time by simulation and experiments.

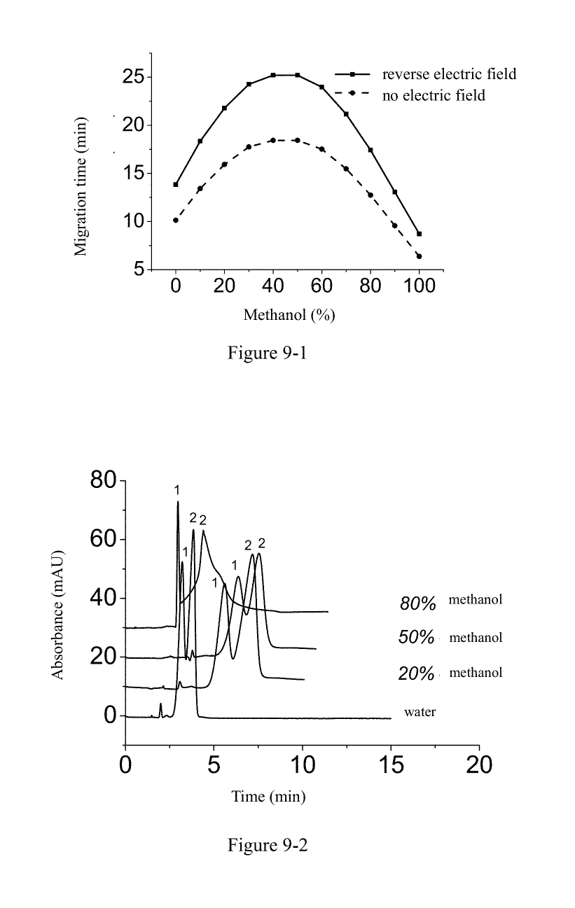

The simulation parameters were set as follows: capillary tube length 60 cm, tube inner diameter 75 μm, reverse separation voltage −10 kV, injection pressure of pumping buffer solution 30 mbar, sample with relative molecular weight 1048, two positive charges and sample equivalent radius 1.2 nm. The buffer solution was water under 25° C. (H=0.89 mPa·S), 10% methanol (η=1.18 mPa·S), 20% methanol (η=1.40 mPa·S), 30% methanol (η=1.56 (H=1.62 mPa·S), 60% methanol (η=1.54 mPa·S), 70% methanol (η=1.36 mPa·S), 80% methanol (η=1.12 mPa·S), 90% methanol (η=0.84 mPa·S), 100% methanol (η=0.56 mPa·S), respectively. The simulation results are shown in FIG. 9-1. It can be seen that with the increase of the methanol content in the aqueous solution, the substance migration time is prolonged and then shortened.

Experimental conditions: The sample was a mixed solution of angiotensin II with a final concentration of 1 mg/mL and 1 mg/mL bradykinin, the capillary tube length is 40 cm, the tube inner diameter was 75 μm. Operation mode: the injection was carried out under an injection pressure of 50 mbar for 5 s, a separation pressure of 50 mbar and a voltage of −20 kV were applied at both ends of the separation channel. The buffer solution was water, 20% methanol, 50% methanol, 80% methanol (containing 0.1% formic acid (w/w)), respectively. The experimental results are shown in FIG. 9-2. It can be seen that with the increase of the methanol content in the aqueous buffer solution, the migration time of the sample increases first and then decreases, and when the methanol content is high, the peak shape is greatly influenced. In this Figure, substance 1 is angiotensin II and substance 2 is bradykinin.

### EXAMPLE 5

The present example analyzes the effect of the charged nature of the sample on the sample migration time by simulation.

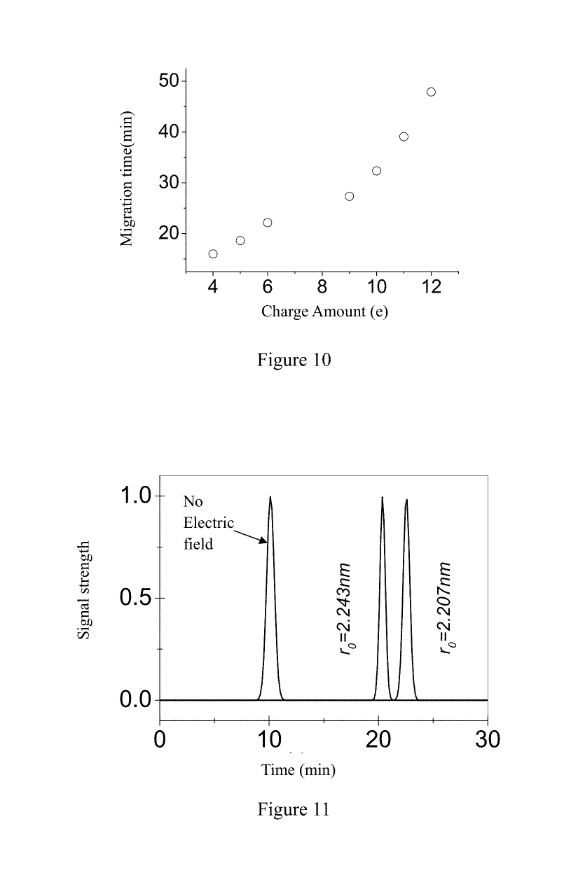

The simulation parameters were set as follows: capillary tube length 60 cm, tube inner diameter 75 m, reverse separation voltage −10 kV, the injection pressure of pumping buffer solution 30 mbar, viscosity coefficient 0.89 mPa·S. The sample has a relative molecular weight of 8600 and equivalent radius of 1.2 nm, with 4, 5, 6, 9, 10, 11 and 12 positive charges, respectively. The simulation results are shown in FIG. 10. It can be seen that with the increase of ion charge, the substance migration time is prolonged. The dissociation of the substance at different pH is different, namely, when the number of charges is different, the peak time will change.

### EXAMPLE 6

The present example analyzes the effect of the geometry of the sample on the sample migration time by simulation.

The simulation parameters were set as follows: capillary tube length 60 cm, tube inner diameter 75 m, reverse separation voltage −10 kV, the injection pressure of pumping buffer solution 30 mbar, viscosity coefficient 0.89 mPa·S. The sample has a relative molecular weight of 8600 and equivalent radius r₀ of 2.243 nm and 2.207 nm, respectively, with 7 positive charges. The simulation results are shown in FIG. 11. It can be seen that as the equivalent radius of the sample becomes larger, the substance migration time will be shortened and the sample will peak in advance.

### EXAMPLE 7

The example analyzes the separation effect of various mixed samples by simulation and experiments, respectively.

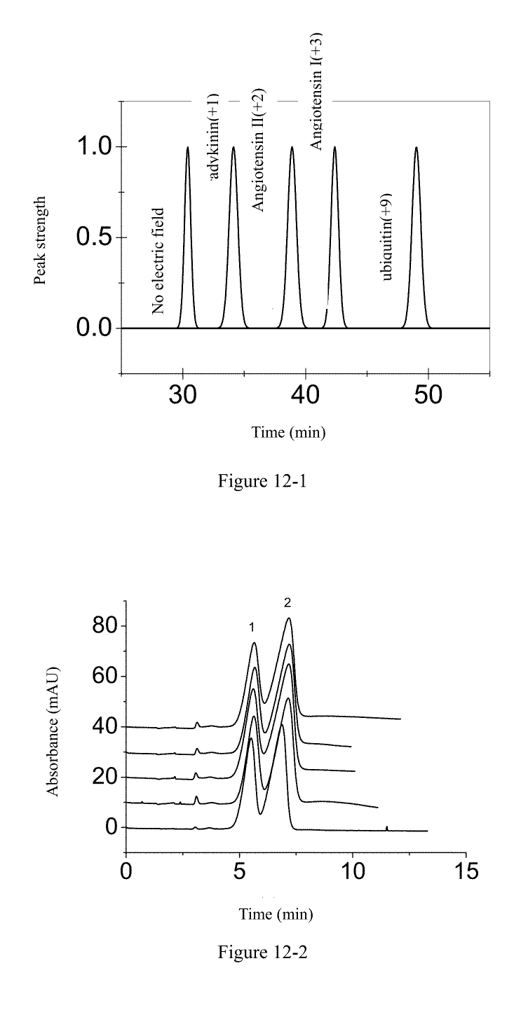

In the simulation, four substances comprising angiotensin I (+3), angiotensin II (+2), bradykinin (+1) and ubiquitin (+9) were selected to carry out simulation of the mixture separation. The simulation conditions were given below: the capillary tube length is 40 cm, the tube inner diameter is 75 μm, the potential is −2 kV, the injection pressure of pumping buffer solution is 10 mbar, and the viscosity coefficient is 0.89 mPa·S. The simulation results are shown in FIG. 12-1, and the separation between the four substances is achieved.

Experimental conditions: The sample is a mixed solution of angiotensin II with a final concentration of 1 mg/mL and 1 mg/mL bradykinin, the buffer solution is 20% methanol aqueous solution (containing 0.1% formic acid (w/w)), the capillary tube length is 40 cm, the tube inner diameter is 75 μm. Operation mode: the injection is carried out under 50 mbar injection pressure for 5 s, 50 mbar separation pressure and −20 kV voltage are applied at both ends of the separation channel, the experimental results are shown in FIG. 12-2, in the Figure, the substance 1 is angiotensin II and substance 2 is bradykinin. It can be seen that separation between the two peptides is achieved and the separation effect is good. Under these conditions, the experiment is repeated five times and the reproducibility is good.

The present invention provides a mobility electrophoresis separation device, a control method thereof and an interface between liquid chromatography and mass spectrometry of, the mobility electrophoresis separation device comprises a separation capillary, a syringe pump, a syringe, a separation electrode and a ground electrode, wherein one end of the capillary is an electrospray tip and the other end is a buffer solution injection end; the syringe pump is connected to the buffer solution injection end; the syringe is connected to the separation capillary at a position close to the syringe pump; the separation electrode is connected to the separation capillary at a position close to the syringe pump; and the ground electrode is connected to the separation capillary at a position close to the electrospray tip. The present invention utilizes a syringe pump to inject buffer solution to rinse the sample in the separation capillary while applying a separate electric field so that the components in the sample are sufficiently separated in the separation capillary to enhance the separation effect with a fast separation rate and the product structure is simple and easy to operate.

Although the invention and its advantages have been described in detail, it should be understood that without departing from the spirit and scope of the appended claims as defined in the present invention that various modifications, substitutions and changes can be made. Moreover, the scope of the present application is not limited to the specific examples of processes, systems, devices, methods and steps described in the specification. A person skilled in the art based on the disclosure of the present invention will readily understand that in accordance with the present invention they can use processes, systems, devices, methods and steps to implement similar functions as the corresponding examples described herein or obtain similar results. Therefore, the appended claims intend to include such processes, systems, devices, methods and steps within their scope.

1. A mobility electrophoresis separation device, comprising:
a separating capillary comprising a first end and a second end, the first end of the separation capillary comprises an electrospray tip and the second end comprising a buffer solution injection end;
a syringe pump connected to the buffer solution injection end;
a syringe connected to the separation capillary at a position close to the syringe pump;
a separation electrode, connected to the syringe pump, or connected to the separation capillary at a position close to the syringe pump; and
a ground electrode, connected to the separation capillary at a position close to the electrospray tip.

2. The mobility electrophoresis separation device according to claim 1, wherein said mobility electrophoresis separation device further comprises:
a first multi-way valve, through which the syringe pump and the syringe are connected to the separation capillary; and
a second multi-way valve, through which the ground electrode is connected to the separation capillary.

3. The mobility electrophoresis separation device according to claim 2, wherein the separation electrode is connected to the separation electrode through the first multi-way valve.

4. The mobility electrophoresis separation device according to claim 2, wherein the mobility electrophoresis separation device further comprises:
an auxiliary capillary, connected to the separation capillary through the second multi-way valve.

5. An interface between liquid chromatography and mass spectrometry, comprising:
the mobility electrophoresis separation device according to claim 1; and
a liquid chromatography apparatus, wherein a sample outlet of the liquid chromatography apparatus is connected to the syringe of the mobility electrophoresis separation device.

6. A method for controlling the mobility electrophoresis separation device according to claim 1, characterized by comprising:
turning on the syringe pump to inject the buffer solution into the separation capillary at a predetermined flushing pressure;
closing the syringe pump after a first time period;
using the syringe to inject the sample into the separation capillary at a predetermined injection pressure;
stopping the injection of the syringe after a second time period, turning on the syringe pump again to inject the buffer solution into the separation capillary at a predetermined separation pressure, applying separation voltage through the separation electrode, and applying spray voltage to the electrospray tip.

7. The method for controlling a mobility electrophoresis separation device according to claim 6, wherein the flushing pressure is in the range of 1 to 1000 mbar, the injection pressure is in the range of 10 to 100 mbar, the separation pressure is in the range of 1 to 200 Mbar, the first time period is in the range of 4 to 6 minutes and the second time period is in the range of 1 to 10 seconds.

8. A mobility electrophoresis separation method, comprising:
creating a flow of a liquid containing different species of ions in a flow channel; and
applying an electric field along the flow channel, thereby separating the different species of ions based on their different traveling velocities in the liquid flow under the applied electric field.

9. The method of claim 8, wherein creating the flow comprises:
injecting a sample solution containing the different species of ions into a first position of the flow channel;
introducing a carrier solution upstream of the first position in the flow channel, thereby causing the carrier solution to flow through the first position and carrying the different species of ions in the sample solution downstream from the first position.

10. The method of claim 9, wherein introducing the buffer solution is performed at a predetermined pressure.

11. The method of claim 8, wherein the flow channel is a capillary channel.
